(19) United States
(12) Patent Application Publication
(10) Pub. No.: US 2025/0245757 A1
(43) Pub. Date: Jul. 31, 2025

(54) Data Processing System and Method

(71) Applicant: HB Innovations, LLC, Washington, MI (US)

(72) Inventor: Michael Nicholas Towle, Lake Orion, MI (US)

(21) Appl. No.: 19/037,922

(22) Filed: Jan. 27, 2025

Related U.S. Application Data
(60) Provisional application No. 63/625,571, filed on Jan. 26, 2024.

(51) Int. Cl. G06Q 40/12; G06Q 20/10; G06Q 20/14

(52) U.S. Cl. CPC G06Q 40/125; G06Q 20/102; G06Q 20/14

(57) ABSTRACT

A method, computer program product, and computing system for: interfacing an intermediate platform with a practice management system; interfacing the intermediate platform with an accounting system; obtaining, on the intermediate platform, payment information from the practice management system, wherein this payment information concerns one or more contracts administered on the practice management system; and providing the payment information to the accounting system so that AR inflows defined within the payment information can be acknowledged for GAAP adherence purposes.

### PRIORITY APPLICATION

This application claims the benefit of U.S. Provisional Application No. 63/625,571, filed on 26 Jan. 2024, the entire contents of which are herein incorporated by reference.

### TECHNICAL FIELD

This disclosure relates to data transfer systems and methods and, more particularly, to systems and methods that transfer data between disparate systems.

### BACKGROUND

Adhering to GAAP (Generally Accepted Accounting Principles) is crucial for managing an orthodontics practice where patients often make monthly payments, as it ensures financial accuracy, transparency, and compliance with accounting standards. GAAP provides a standardized framework for recording and reporting financial transactions, which is especially important for practices managing long-term payment plans and deferred revenue.

In an orthodontics practice, patients typically pay for treatments over time, with an initial upfront payment followed by monthly installments. GAAP principles, such as the revenue recognition principle, require practices to recognize revenue only when the associated services are performed, rather than when payments are received. This is particularly relevant for orthodontic treatments, which often span several months or years. Adhering to these standards ensures that revenue is recorded proportionally over the treatment period, accurately reflecting the practice's financial position and performance.

The matching principle is another key aspect of GAAP, which mandates that expenses incurred to deliver services be matched with the revenue earned from those services. For example, costs associated with creating aligners, conducting diagnostic tests, or providing ongoing care must be accounted for in the same period as the corresponding revenue. This alignment helps the practice track profitability more effectively and avoid overstating or understating financial results.

Following GAAP also helps orthodontic practices maintain transparency and build trust with stakeholders, including investors, lenders, and regulators. Accurate financial reporting is essential for securing loans, attracting investment, and complying with tax regulations. Additionally, adhering to GAAP can reduce the risk of audits, penalties, or legal issues that may arise from improper financial practices.

For orthodontic practices with multiple patients on payment plans, GAAP standards facilitate better financial management by ensuring that payments are categorized appropriately, such as recognizing upfront payments as deferred revenue until the corresponding services are delivered. This structured approach helps practices manage cash flow, plan for future expenses, and make informed business decisions.

In summary, adhering to GAAP in an orthodontics practice that relies on monthly payments ensures accurate financial reporting, compliance with regulations, and a clear representation of the practice's financial health. It supports better decision-making, fosters trust with stakeholders, and helps the practice achieve long-term financial stability.

### SUMMARY OF DISCLOSURE

### AR Platform

In one implementation, a computer-implemented method is executed on a computing device and includes: interfacing an intermediate platform with a practice management system; interfacing the intermediate platform with an accounting system; obtaining, on the intermediate platform, payment information from the practice management system, wherein this payment information concerns one or more contracts administered on the practice management system; and providing the payment information to the accounting system so that AR inflows defined within the payment information can be acknowledged for GAAP adherence purposes.

One or more of the following features may be included. Interfacing an intermediate platform with a practice management system may include: configuring a first application program interface on the practice management system to enable interfacing with the intermediate platform. Interfacing the intermediate platform with an accounting system may include: configuring a second application program interface on the accounting system to enable interfacing with the intermediate platform. The practice management system may include: an orthodontics practice management system. The payment information provided to the accounting system may be utilizable to reconcile AR inflows defined within the payment information with cash inflows defined within an external bank account. Obtaining, on the intermediate platform, payment information from the practice management system may include: periodically obtaining, on the intermediate platform, payment information from the practice management system. A rules database may be maintained that defines a plurality of contract types and one or more GAAP-adhering waterfalling rules concerning the plurality of contract types. The payment information obtained from the practice management system may be processed to remove Personally Identifiable Information (PII) from the payment information. A reconciliation procedure may be effectuated to ensure that changes made within the practice management system are replicated onto the accounting system. A reconciliation procedure may be effectuated to ensure that changes made within the accounting system are replicated onto the practice management system.

In another implementation, a computer program product resides on a computer readable medium and has a plurality of instructions stored on it. When executed by a processor, the instructions cause the processor to perform operations including: interfacing an intermediate platform with a practice management system; interfacing the intermediate platform with an accounting system; obtaining, on the intermediate platform, payment information from the practice management system, wherein this payment information concerns one or more contracts administered on the practice management system; and providing the payment information to the accounting system so that AR inflows defined within the payment information can be acknowledged for GAAP adherence purposes.

One or more of the following features may be included. Interfacing an intermediate platform with a practice management system may include: configuring a first application program interface on the practice management system to enable interfacing with the intermediate platform. Interfacing the intermediate platform with an accounting system may include: configuring a second application program interface on the accounting system to enable interfacing with the intermediate platform. The practice management system may include: an orthodontics practice management system. The payment information provided to the accounting system may be utilizable to reconcile AR inflows defined within the payment information with cash inflows defined within an external bank account. Obtaining, on the intermediate platform, payment information from the practice management system may include: periodically obtaining, on the intermediate platform, payment information from the practice management system. A rules database may be maintained that defines a plurality of contract types and one or more GAAP-adhering waterfalling rules concerning the plurality of contract types. The payment information obtained from the practice management system may be processed to remove Personally Identifiable Information (PII) from the payment information. A reconciliation procedure may be effectuated to ensure that changes made within the practice management system are replicated onto the accounting system. A reconciliation procedure may be effectuated to ensure that changes made within the accounting system are replicated onto the practice management system.

In another implementation, a computing system including a processor and memory is configured to perform operations including: interfacing an intermediate platform with a practice management system; interfacing the intermediate platform with an accounting system; obtaining, on the intermediate platform, payment information from the practice management system, wherein this payment information concerns one or more contracts administered on the practice management system; and providing the payment information to the accounting system so that AR inflows defined within the payment information can be acknowledged for GAAP adherence purposes.

One or more of the following features may be included. Interfacing an intermediate platform with a practice management system may include: configuring a first application program interface on the practice management system to enable interfacing with the intermediate platform. Interfacing the intermediate platform with an accounting system may include: configuring a second application program interface on the accounting system to enable interfacing with the intermediate platform. The practice management system may include: an orthodontics practice management system. The payment information provided to the accounting system may be utilizable to reconcile AR inflows defined within the payment information with cash inflows defined within an external bank account. Obtaining, on the intermediate platform, payment information from the practice management system may include: periodically obtaining, on the intermediate platform, payment information from the practice management system. A rules database may be maintained that defines a plurality of contract types and one or more GAAP-adhering waterfalling rules concerning the plurality of contract types. The payment information obtained from the practice management system may be processed to remove Personally Identifiable Information (PII) from the payment information. A reconciliation procedure may be effectuated to ensure that changes made within the practice management system are replicated onto the accounting system. A reconciliation procedure may be effectuated to ensure that changes made within the accounting system are replicated onto the practice management system.

The details of one or more implementations are set forth in the accompanying drawings and the description below. Other features and advantages will become apparent from the description, the drawings, and the claims.

### BRIEF DESCRIPTION OF THE DRAWINGS

FIG. 1 is a diagrammatic view of a distributed computing network including a computing device that executes a data transfer process according to an implementation of the present disclosure;

FIG. 2 is a diagrammatic view of a data transfer instance effectuated by the data transfer process of FIG. 1 according to an implementation of the present disclosure;

FIG. 3 is a flowchart of an implementation of the data transfer process of FIG. 1 according to an implementation of the present disclosure; and

FIG. 4 is a flowchart of another implementation of the data transfer process of FIG. 1 according to an implementation of the present disclosure.

Like reference symbols in the various drawings indicate like elements.

### DETAILED DESCRIPTION OF THE PREFERRED EMBODIMENTS

### System Overview

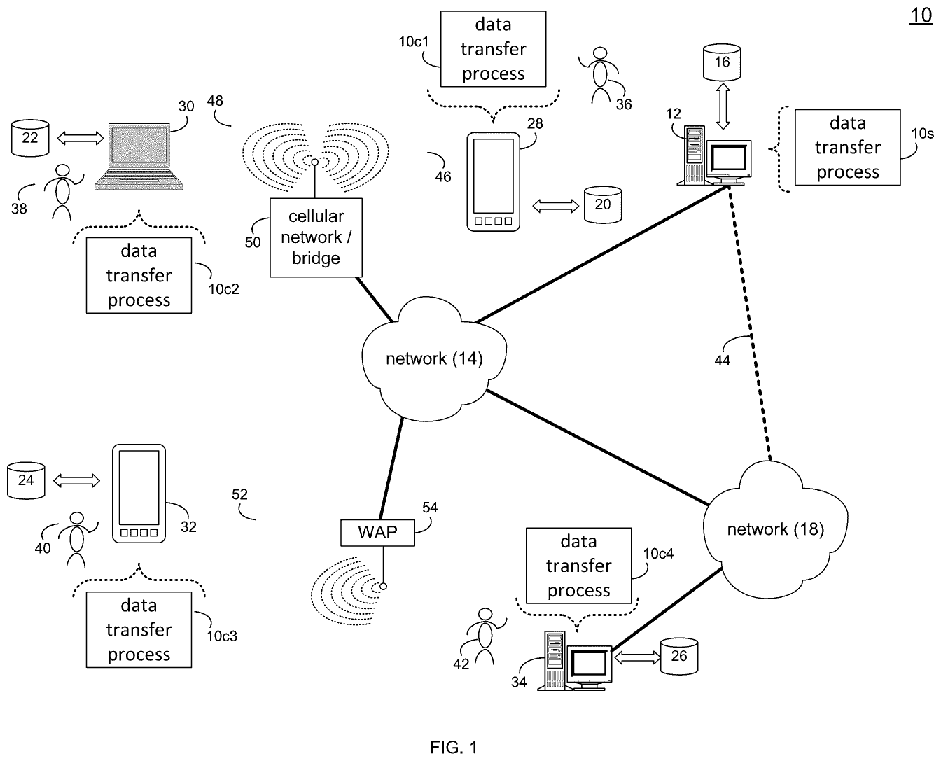

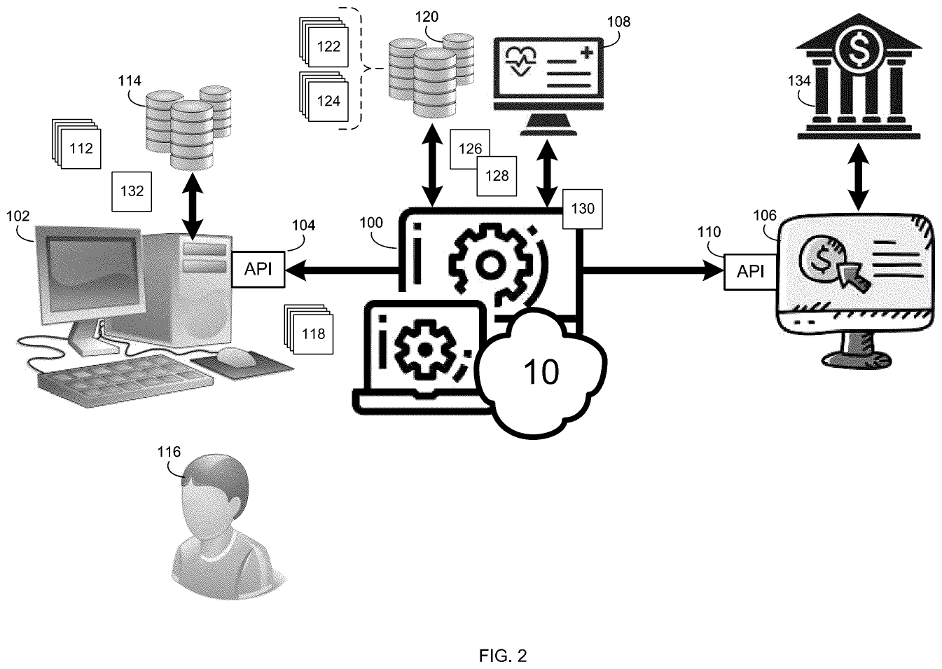

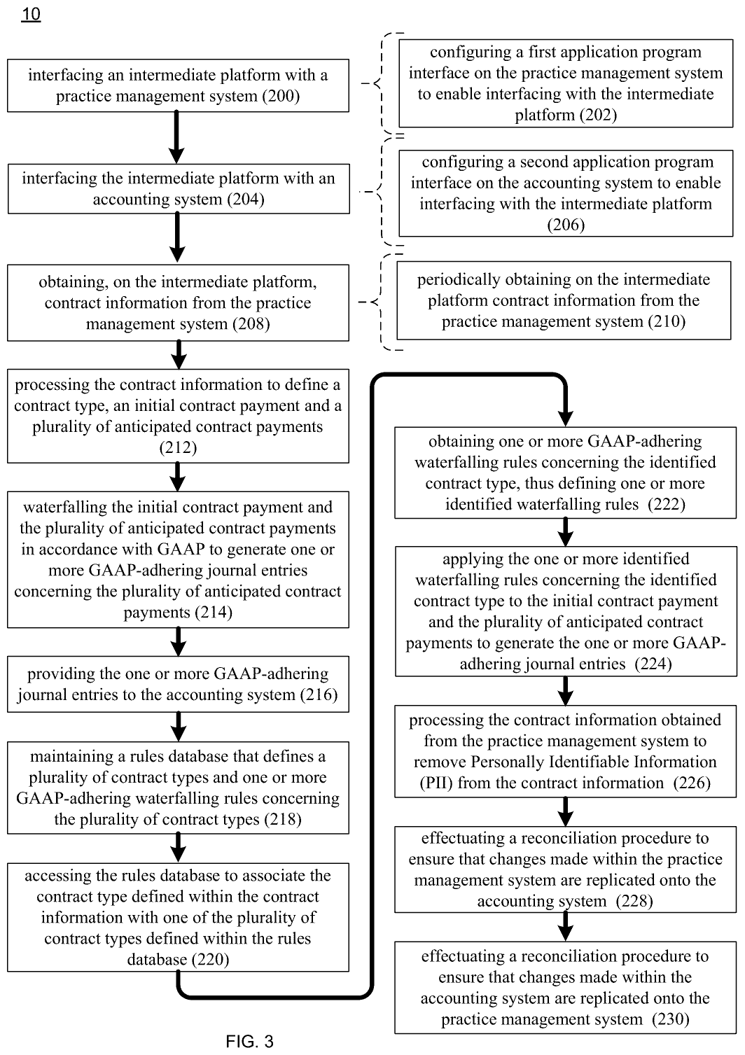

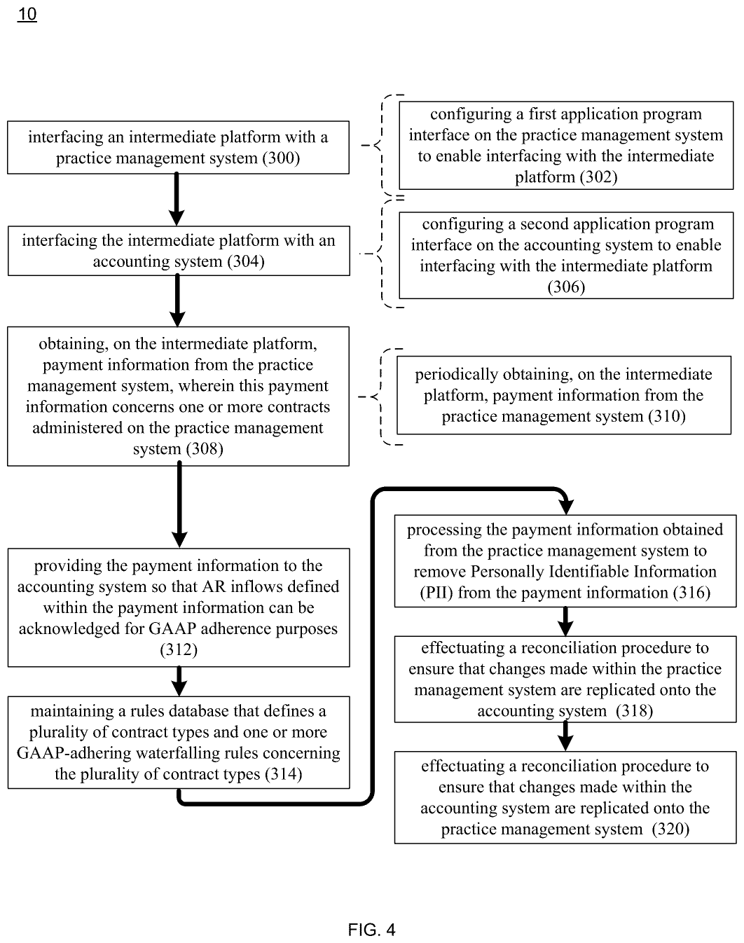

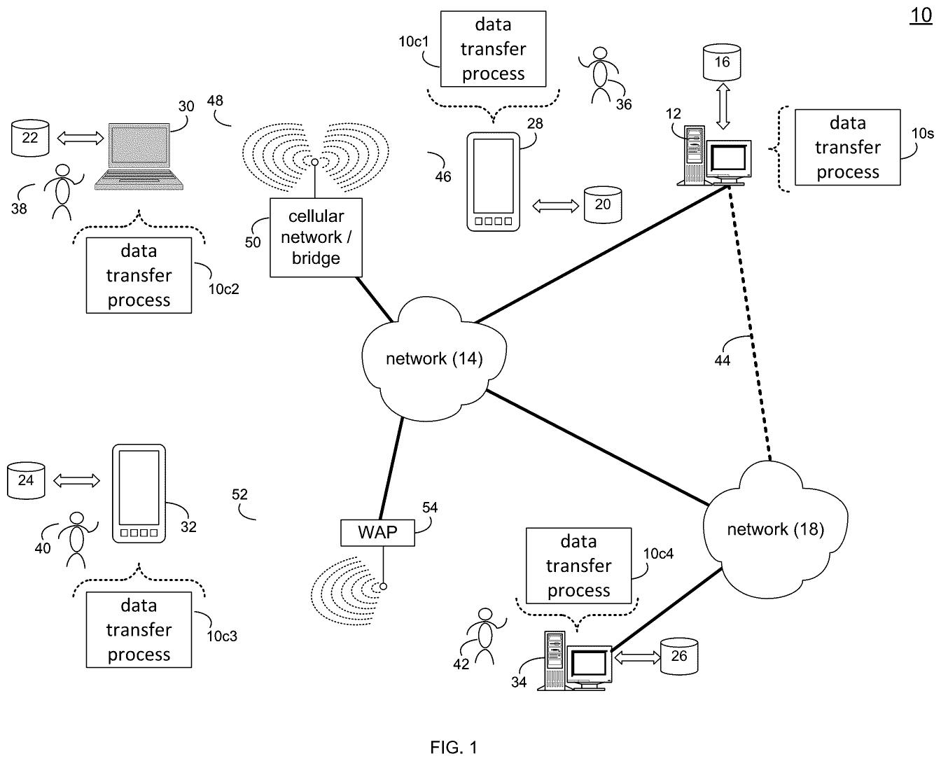

Referring to FIG. 1, there is shown data transfer process 10. As will be discussed below in greater detail, data transfer process 10 may be configured to match people in need of assistance with paying their medical expenses with charitable funds that offer such assistance.

Data transfer process 10 may be implemented as a server-side process, a client-side process, or a hybrid server-side/client-side process. For example, data transfer process 10 may be implemented as a purely server-side process via data transfer process 10s. Alternatively, data transfer process 10 may be implemented as a purely client-side process via one or more of data transfer process 10c1, data transfer process 10c2, data transfer process 10c3, and data transfer process 10c4. Alternatively still, data transfer process 10 may be implemented as a hybrid server-side/client-side process via data transfer process 10s in combination with one or more of data transfer process 10c1, data transfer process 10c2, data transfer process 10c3, and data transfer process 10c4. Accordingly, data transfer process 10 as used in this disclosure may include any combination of data transfer process 10s, data transfer process 10c1, data transfer process 10c2, data transfer process 10c3, and data transfer process 10c4.

Data transfer process 10s may be a server application and may reside on and may be executed by computing device 12, which may be connected to network 14 (e.g., the Internet or a local area network). Examples of computing device 12 may include, but are not limited to: a personal computer, a laptop computer, a personal digital assistant, a data-enabled cellular telephone, a notebook computer, a television with one or more processors embedded therein or coupled thereto, a cable/satellite receiver with one or more processors embedded therein or coupled thereto, a server computer, a series of server computers, a mini computer, a mainframe computer, or a dedicated network device.

The instruction sets and subroutines of data transfer process 10s, which may be stored on storage device 16 coupled to computing device 12, may be executed by one or more processors (not shown) and one or more memory architectures (not shown) included within computing device 12. Examples of storage device 16 may include but are not limited to: a hard disk drive; a tape drive; an optical drive; a RAID device; a random access memory (RAM); a read-only memory (ROM); and all forms of flash memory storage devices.

Network 14 may be connected to one or more secondary networks (e.g., network 18), examples of which may include but are not limited to: a local area network; a wide area network; or an intranet, for example.

Examples of data transfer processes 10c1, 10c2, 10c3, 10c4 may include but are not limited to a web browser, a game console user interface, or a specialized application (e.g., an application running on e.g., the Android tm platform or the iPhone tm platform). The instruction sets and subroutines of roadside assistance applications 10c1, 10c2, 10c3, 10c4, which may be stored on storage devices 20, 22, 24, 26 (respectively) coupled to client electronic devices 28, 30, 32, 34 (respectively), may be executed by one or more processors (not shown) and one or more memory architectures (not shown) incorporated into client electronic devices 28, 30, 32, 34 (respectively). Examples of storage devices 20, 22, 24, 26 may include but are not limited to: hard disk drives; tape drives; optical drives; RAID devices; random access memories (RAM); read-only memories (ROM), and all forms of flash memory storage devices.

Examples of client electronic devices 28, 30, 32, 34 may include, but are not limited to, data-enabled, cellular telephone 28, laptop computer 30, personal digital assistant 32, personal computer 34, a notebook computer (not shown), a server computer (not shown), a gaming console (not shown), a smart television (not shown), and a dedicated network device (not shown). Client electronic devices 28, 30, 32, 34 may each execute an operating system, examples of which may include but are not limited to Microsoft Windows tm, Android tm, WebOS tm, iOS tm, Redhat Linux tm, or a custom operating system.

Additionally/alternatively, virtual desktops and virtual machines may be utilized to access data transfer process 10. As is known in the art, a virtual machine (VM) is a software-based emulation of a physical computer, allowing multiple operating systems to run simultaneously on a single physical machine. It functions as an isolated environment that mimics the hardware of a computer, enabling users to install and operate operating systems and applications as if they were on a dedicated physical device. Virtual machines are managed by a hypervisor, which allocates resources such as CPU, memory, and storage to each VM while ensuring they operate independently from one another. This technology is widely used for software testing, running legacy applications, server consolidation, and creating secure environments. By abstracting hardware, virtual machines provide flexibility, scalability, and cost efficiency in both development and production settings. As is known in the art, a virtual desktop is a computing environment that allows users to access a desktop operating system and applications hosted on a remote server rather than a local device. Delivered through technologies like Virtual Desktop Infrastructure (VDI) or cloud services, a virtual desktop provides a consistent, customizable workspace accessible from various devices, including laptops, tablets, and smartphones. This setup enables centralized management of desktops, ensuring that updates, security, and backups are handled on the server side, enhancing IT efficiency and reducing maintenance costs. Virtual desktops are widely used for remote work, as they allow employees to securely access their work environment from anywhere, while businesses benefit from enhanced data security, scalability, and reduced hardware dependency.

Users 36, 38, 40, 42 may access data transfer process 10 directly through network 14 or through secondary network 18. Further, data transfer process 10 may be connected to network 14 through secondary network 18, as illustrated with link line 44.

The various client electronic devices (e.g., client electronic devices 28, 30, 32, 34) may be directly or indirectly coupled to network 14 (or network 18). For example, data-enabled, cellular telephone 28 and laptop computer 30 are shown wirelessly coupled to network 14 via wireless communication channels 46, 48 (respectively) established between data-enabled, cellular telephone 28, laptop computer 30 (respectively) and cellular network/bridge 50, which is shown directly coupled to network 14. Further, personal digital assistant 32 is shown wirelessly coupled to network 14 via wireless communication channel 52 established between personal digital assistant 32 and wireless access point (i.e., WAP) 54, which is shown directly coupled to network 14. Additionally, personal computer 34 is shown directly coupled to network 18 via a hardwired network connection.

WAP 54 may be, for example, an IEEE 802.11a, 802.11b, 802.11g, 802.11n, Wi-Fi, and/or Bluetooth device that is capable of establishing wireless communication channel 52 between personal digital assistant 32 and WAP 54. As is known in the art, IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation, for example. As is known in the art, Bluetooth is a telecommunications industry specification that allows e.g., mobile phones, computers, and personal digital assistants to be interconnected using a short-range wireless connection.

### Generally Accepted Accounting Principals (GAAP):

GAAP, or Generally Accepted Accounting Principles, is a comprehensive set of accounting standards, principles, and procedures established to guide organizations in the preparation and presentation of their financial statements in the United States. It serves as the foundation for financial reporting, ensuring consistency, comparability, and transparency across different organizations and industries. By providing a standardized framework, GAAP helps create a common language for financial information, which is crucial for investors, regulators, analysts, and other stakeholders who rely on accurate and reliable data to make informed decisions.

The principles under GAAP include key concepts such as the revenue recognition principle, which dictates that revenue should be recognized when it is earned and realizable; the matching principle, which requires that expenses be recorded in the same period as the revenues they help generate; the full disclosure principle, which ensures that all relevant financial information is presented in financial reports; and the cost principle, which states that assets should be recorded based on their original purchase cost rather than their current market value. These principles form the backbone of GAAP, guiding accountants in handling complex financial transactions and reporting them accurately.

GAAP is primarily developed and maintained by the Financial Accounting Standards Board (FASB), an independent organization that sets accounting standards in the United States. Compliance with GAAP is mandatory for publicly traded companies as per the regulations of the Securities and Exchange Commission (SEC). This requirement ensures that companies adhere to a consistent set of rules, which is vital for maintaining investor confidence and ensuring fair and transparent markets. Private companies, while not legally required to follow GAAP, often choose to do so to present credible financial reports to lenders, investors, and other stakeholders.

One of the defining characteristics of GAAP is its rules-based nature, which provides detailed guidelines and specific instructions for a wide range of accounting scenarios. This can help ensure precision but can also result in complexities and rigidities in its application. GAAP is particularly significant in promoting comparability across financial statements, allowing stakeholders to evaluate the performance of different organizations on a level playing field.

Ultimately, GAAP plays a critical role in fostering trust in financial reporting. It ensures that financial statements are not only accurate and reliable but also consistent and transparent, thereby safeguarding the interests of various stakeholders and supporting the overall integrity of financial markets. Its standardized approach reduces the risk of fraud, misrepresentation, and errors, making it an indispensable part of the accounting and financial reporting landscape in the United States.

### Waterfalling and GAAP?

Waterfalling is important in GAAP (Generally Accepted Accounting Principles) because it provides a structured and systematic method for allocating payments or resources in compliance with accounting standards. The waterfall approach ensures that financial transactions are recorded accurately and consistently, which is critical for maintaining transparency, meeting reporting obligations, and ensuring the financial statements are prepared in accordance with GAAP.

Under GAAP, revenue recognition and expense matching are governed by specific principles, such as the revenue recognition principle and the matching principle. A waterfall structure supports these principles by prioritizing the allocation of payments to specific obligations in a pre-determined order, ensuring that transactions are accounted for in the correct periods. For example, in contracts with upfront payments and subsequent installments, a waterfall framework helps allocate revenue to different services or deliverables, recognizing income appropriately as those services are performed or obligations are fulfilled. This prevents premature or improper recognition of revenue, which could misrepresent the financial health of an entity.

From a compliance perspective, waterfalling aligns with GAAP's focus on clarity, consistency, and comparability in financial reporting. By detailing how funds flow through a contract and ensuring that obligations are met in priority order, waterfalling reduces the risk of accounting errors, such as double-counting revenue or failing to account for deferred obligations. For example, in subscription-based models or service contracts, a waterfall process ensures that upfront payments are initially allocated to deferred revenue and only recognized as revenue over time as the service is delivered.

In addition, waterfalling is particularly important in areas like multi-element arrangements, where GAAP requires entities to allocate payments across multiple performance obligations in proportion to their standalone selling prices. The waterfall structure ensures that payments are applied correctly, making it easier to demonstrate compliance with standards such as ASC 606 (Revenue from Contracts with Customers), which emphasizes recognizing revenue in line with the transfer of goods or services to customers.

Ultimately, waterfalling enhances financial control, supports proper revenue recognition, and ensures alignment with GAAP principles. It provides the clarity and structure necessary to manage complex contracts and payments while maintaining the accuracy and integrity of financial statements. This approach is particularly valuable in industries with long-term contracts or staged payments, as it ensures that obligations are prioritized and accounted for in a manner that is fair, transparent, and compliant with GAAP.

### Waterfalling, GAAP and Practice Management

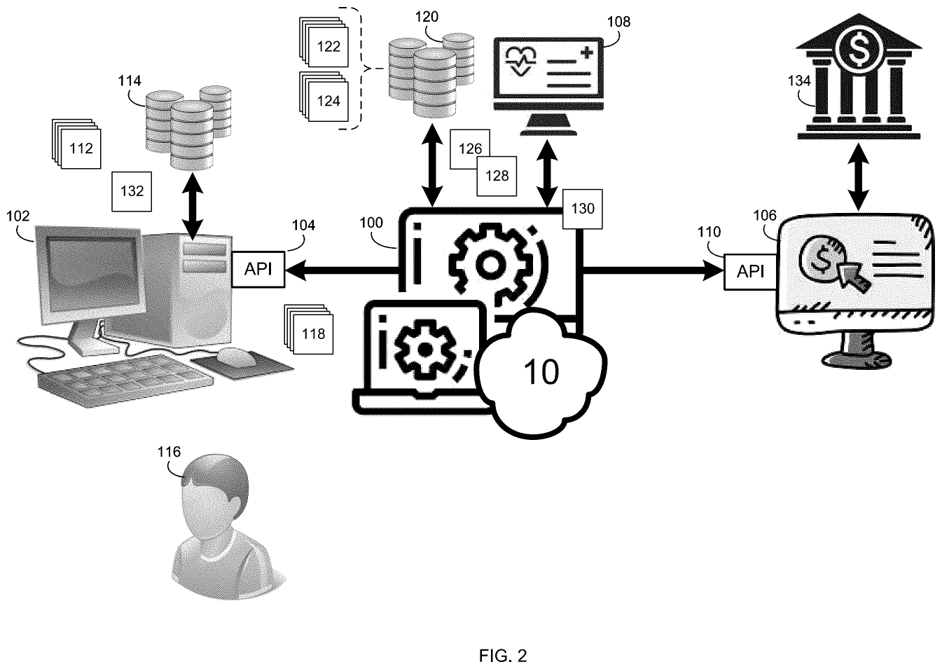

Referring also to FIG. 2, while the following discussion concerns an intermediate platform (e.g., intermediate platform 100) interfaced with a practice management system (e.g., practice management system 102) that is used by orthodontics practice, this is for illustrative purposes only and is not intended to be a limitation of this disclosure. Specifically, the below disclosure may be applicable to any industry in which adherence to GAAP standards is of importance.

In the orthodontics industry, waterfalling contracts refer to a structured and prioritized method of allocating payments made by patients, which typically include an initial upfront payment followed by recurring monthly payments over the course of treatment. This payment structure ensures that financial obligations tied to the treatment are addressed systematically, with high-priority costs covered first before funds flow to subsequent expenses. The upfront payment often covers essential, non-refundable costs such as lab fees for creating custom aligners or braces, initial diagnostic services like X-rays and impressions, and administrative fees for setting up the patient's account and treatment plan. These costs are critical to initiating treatment, and securing them upfront mitigates financial risk for the orthodontic practice.

Subsequent monthly payments are distributed in a pre-determined order, typically covering ongoing professional services like periodic adjustments, follow-up appointments, and patient support. Depending on the contract, these payments may also include financing fees if the patient has opted for a payment plan, as well as deferred costs for materials or supplies used later in the treatment. The waterfall structure ensures that each payment is systematically applied to its designated category, creating transparency for both the patient and the provider. For example, if a patient's treatment costs $6,000, with a $1,200 upfront payment and $200 monthly payments over 24 months, the upfront payment might first cover lab fees, administrative setup, and initial services, while the monthly payments are allocated to ongoing care and any financing-related expenses.

This approach offers several benefits to orthodontic practices. First, the upfront payment provides financial security by covering immediate, high-priority costs, allowing the practice to avoid out-of-pocket expenses. Second, the waterfall structure creates a predictable and organized system for managing payments, ensuring that the practice remains financially stable and that resources are allocated efficiently. Additionally, it improves communication and trust with patients by clearly outlining how their payments are applied to the services they receive. For patients, spreading the cost over monthly installments makes treatment more affordable and accessible.

Despite these advantages, the waterfall approach can present challenges. If a patient defaults on their monthly payments, the practice may face a shortfall in covering downstream obligations, especially for services already rendered. Practices must implement effective systems for tracking payments, allocating funds, and addressing potential non-payment issues. Additionally, administrative complexity may increase, particularly for practices handling a large volume of patients on diverse payment plans.

Overall, waterfalling contracts provide orthodontic practices with a robust financial framework that aligns patient payments with service delivery while safeguarding essential costs. This structure not only ensures the practice remains financially sustainable but also enhances patient satisfaction by offering clarity and flexibility in payment options. With proper management, it becomes a reliable method to balance affordability for patients and operational efficiency for providers.

Waterfalling Platform

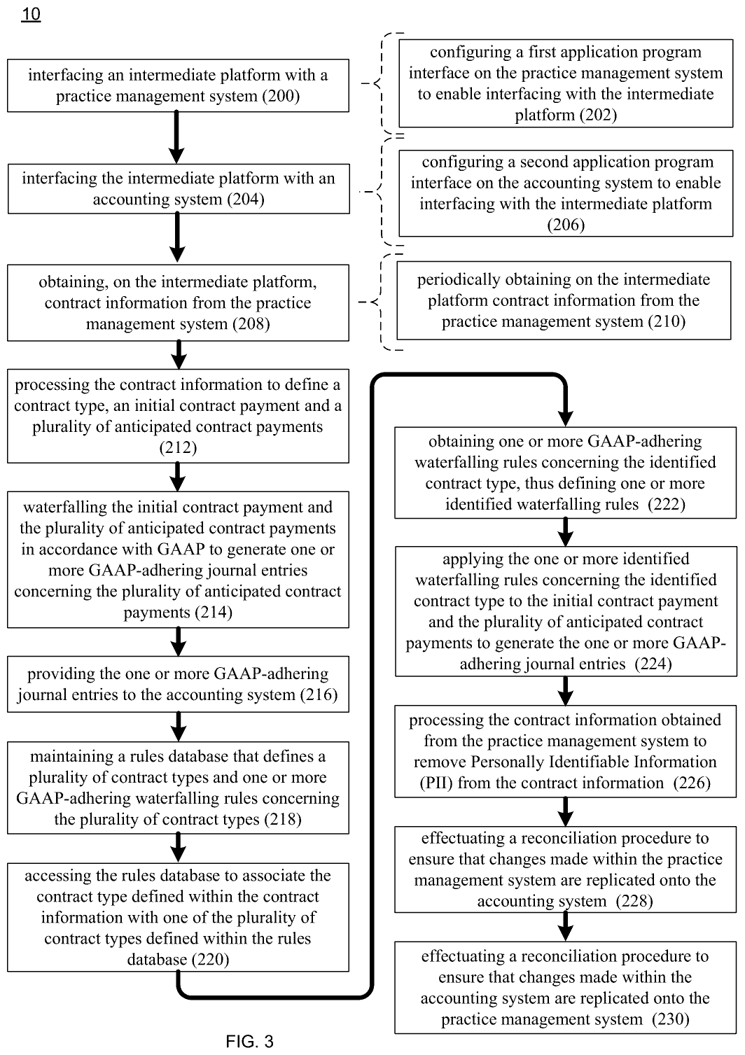

Referring also to FIG. 3, data transfer process 10 may interface 200 an intermediate platform (e.g., intermediate platform 100) with a practice management system (e.g., practice management system 102). This intermediate platform (e.g., intermediate platform 100) may be the platform that effectuates data transfer process 10. And as discussed above, data transfer process 10 as used in this disclosure may include any combination of data transfer process 10s, data transfer process 10c1, data transfer process 10c2, data transfer process 10c3, and data transfer process 10c4.

A practice management system (e.g., practice management system 102) is a software platform designed to help healthcare practices, including medical practices, efficiently manage their administrative, financial, and operational tasks. It serves as a centralized tool for handling various aspects of practice management, such as patient scheduling, appointment reminders, billing, insurance claims processing, and record-keeping. By streamlining these processes, a PMS enables practices to enhance productivity, reduce administrative burden, and provide a seamless experience for both staff and patients.

By centralizing and automating many routine tasks, a PMS helps practices improve efficiency, minimize errors, and focus more on delivering high-quality patient care. It also ensures better financial management by providing tools for accurate billing, tracking outstanding balances, and maintaining compliance with healthcare and accounting regulations.

An example of such a practice management system (e.g., practice management system 102) may include but is not limited to: an orthodontics practice management system.

As is known in the art, an orthodontics practice is a specialized healthcare facility focused on diagnosing, preventing, and treating dental and facial irregularities, primarily related to the alignment of teeth and jaws. Orthodontists, the dental specialists who operate these practices, provide treatments to correct issues such as misaligned teeth (malocclusion), overcrowding, gaps, overbites, underbites, and crossbites. The primary goal of an orthodontics practice is to improve the functionality, aesthetics, and overall health of a patient's smile.

Orthodontic practices typically offer a range of treatments tailored to the unique needs of each patient. These treatments may include traditional braces, clear aligners (such as Invisalign), retainers, and other orthodontic appliances. Depending on the severity of the condition, treatment plans can span several months to years and often require periodic adjustments and monitoring. Many orthodontic practices also cater to patients of all ages, from children and teenagers to adults seeking corrective treatments.

In addition to providing clinical care, an orthodontics practice handles administrative tasks such as patient scheduling, billing, financial contract management, and coordination with insurance providers. These practices often rely on tools like practice management systems and accounting software to streamline operations and enhance patient experiences. By combining specialized expertise with advanced technology, orthodontics practices play a critical role in improving oral health, boosting self-confidence, and enhancing overall quality of life for their patients.

Accordingly, a practice management system (PMS) in the context of an orthodontics practice is a specialized software platform designed to help manage the administrative, financial, and operational aspects of the practice efficiently. It serves as a centralized hub for coordinating patient care, scheduling, billing, and communication, allowing the orthodontist and staff to streamline daily tasks while maintaining a high level of patient service.

For an orthodontics practice, a PMS typically includes features tailored to the unique needs of orthodontic treatment, such as patient intake, appointment scheduling, treatment tracking, and progress monitoring. It also manages billing and financial contracts, which is crucial in orthodontics due to the frequent use of long-term payment plans. The system may automate reminders for appointments, generate financial reports, and assist with submitting insurance claims, ensuring smoother operations and improved cash flow.

Additionally, an orthodontic PMS often integrates with imaging systems, diagnostic tools, and patient engagement platforms, providing a seamless workflow for both clinical and administrative tasks. By using a PMS, orthodontic practices can reduce manual errors, improve patient experiences, and optimize overall efficiency, allowing the orthodontist and staff to focus more on delivering quality care and achieving better patient outcomes.

An example of such an orthodontics practice management system may include but is not limited to the Dolphin Practice Management System. The Dolphin Practice

Management System is a specialized software solution designed to streamline the operations of orthodontic and dental practices, improving patient care and practice efficiency. It offers a comprehensive suite of tools for managing patient information, appointments, billing, treatment planning, and communications all within one integrated platform. The system simplifies patient management by storing and organizing medical histories, treatment progress, and notes, enabling personalized care. It also features an efficient appointment scheduling system that reduces errors, optimizes the clinic's calendar, and includes reminders to ensure patients do not miss appointments.

Dolphin's treatment planning tools allow orthodontists to create customized treatment plans, track progress, and incorporate digital imaging, such as X-rays and 3D scans, into the process. Additionally, it streamlines billing and insurance processes, simplifying claims and payment tracking. The system includes powerful reporting and analytics capabilities, providing insights into practice performance, financial data, and patient demographics. Patient communication is also enhanced with tools for sending appointment reminders and follow-up messages through various channels, such as email and text, helping maintain better engagement. Overall, the Dolphin Practice Management System is an essential tool for orthodontic and dental practices, offering a centralized solution for managing key aspects of the practice while improving efficiency, reducing administrative burdens, and delivering better patient care.

When interfacing 200 an intermediate platform (e.g., intermediate platform 100) with a practice management system (e.g., practice management system 102), data transfer process 10 may configure 202 a first application program interface (e.g., first API 104) on the practice management system (e.g., practice management system 102) to enable interfacing 200 with the intermediate platform (e.g., intermediate platform 100).

An API (Application Programming Interface), in the context of a practice management system (PMS), is a set of tools and protocols that enable different software applications to communicate and interact with the PMS. APIs act as a bridge, allowing seamless data exchange and integration between the PMS and external systems, such as electronic health records (EHR), billing software, diagnostic tools, and patient portals. By enabling these connections, APIs facilitate the automation of tasks like updating patient records, sending appointment reminders, and processing payments, reducing administrative burdens and minimizing errors. They also provide real-time access to critical data, ensuring users have up-to-date information for decision-making. For example, in an orthodontic practice, an API could integrate the PMS with imaging software, allowing X-ray or MRI results to be directly linked to patient profiles, streamlining workflows. APIs also support customization, enabling practices to tailor their PMS by connecting it to specific tools or systems unique to their operations. Additionally, APIs enhance scalability by allowing new integrations as the practice grows or adopts new technologies, eliminating the need for significant system overhauls. Ultimately, APIs improve efficiency, accuracy, and interoperability within the practice, empowering orthodontic teams to deliver better care by creating a connected and modern digital ecosystem.

Data transfer process 10 may interface 204 the intermediate platform (e.g., intermediate platform 100) with an accounting system (e.g., accounting system 106).

An accounting system (e.g., accounting system 106) in the context of an orthodontic practice is specialized software designed to manage the financial and accounting functions of the practice efficiently. It provides tools to handle tasks such as bookkeeping, revenue tracking, expense management, billing, payroll, and financial reporting. By automating these processes, an accounting system (e.g., accounting system 106) helps orthodontic practices maintain accurate financial records, ensure compliance with regulations, and make informed decisions to improve financial health.

Orthodontic practices often deal with complex billing scenarios, including insurance claims, patient co-pays, and installment payment plans for long-term treatments. An accounting system (e.g., accounting system 106) simplifies these processes by integrating with the practice management system (e.g., practice management system 102) or electronic health record system (e.g., EHR system 108) to sync patient financial data. An accounting system (e.g., accounting system 106) can track revenue from multiple sources, categorize expenses, and automate billing workflows, reducing administrative burdens and minimizing errors.

Additionally, an accounting system (e.g., accounting system 106) provides detailed financial reporting and analytics, allowing the practice to monitor its profitability, cash flow, and overall financial performance. This is especially important for orthodontic practices, which may have high operating costs due to specialized equipment, facilities, and staffing needs. The system (e.g., accounting system 106) can generate customized reports to help practitioners identify areas of improvement, plan budgets, and forecast future revenues.

Many accounting systems also include features to manage payroll, ensuring staff salaries, bonuses, and benefits are processed accurately and on time. They may also assist with tax preparation and compliance, helping the practice meet regulatory requirements and avoid penalties.

An example of such an accounting system may include but is not limited to the Sage Impact System. Sage Impact is a cloud-based platform developed by Sage Group, designed to help accounting professionals streamline their workflow, enhance productivity, and manage client relationships more effectively. The platform provides a centralized dashboard where users can access key tools, financial data, and client information, allowing for a more organized and efficient approach to accounting tasks. It also offers collaboration tools that enable seamless communication between accounting teams and their clients, improving task management and ensuring deadlines are met. Sage Impact integrates with other Sage products, such as Sage Accounting, Sage Payroll, and Sage HR, creating a cohesive ecosystem that enhances functionality and efficiency.

Sage Impact includes practice management tools for client portfolio management, invoicing, billing, and scheduling, all of which help accountants stay on top of their workload. It also offers educational resources to keep users informed about industry developments and compliance updates. Security is a key focus, with the platform adhering to high standards to protect sensitive financial data. Overall, Sage Impact is a valuable tool for improving accounting practices by increasing efficiency, supporting better client management, and ensuring compliance with evolving regulations.

When interfacing 204 the intermediate platform (e.g., intermediate platform 100) with an accounting system (e.g., accounting system 106), data transfer process 10 may configure 206 a second application program interface (e.g., second API 110) on the accounting system (e.g., accounting system 106) to enable interfacing 204 with the intermediate platform (e.g., intermediate platform 100).

An API (Application Programming Interface), in the context of interfacing with an accounting system (e.g., accounting system 106), is a set of protocols and tools that enable different software applications to communicate with the accounting system. It acts as a bridge, allowing data to flow seamlessly between the accounting software and other systems, such as practice management systems, payment processing platforms, customer relationship management (CRM) tools, or payroll systems. This integration facilitates automated data sharing and enhances the efficiency and accuracy of financial processes.

The use of APIs in accounting systems simplifies tasks like generating reports, reconciling accounts, and managing tax compliance by integrating data from multiple sources. APIs also make it possible to customize workflows, as practices can choose specific third-party applications to meet their unique needs. For example, an orthodontic practice might use an API to link its accounting software to a specialized inventory management tool for tracking high-value medical equipment.

Data transfer process 10 may obtain 208, on the intermediate platform (e.g., intermediate platform 100), contract information (e.g., contract information 112) from the practice management system (e.g., practice management system 102). Such contract information (e.g., contract information 112) may be obtained from a database (e.g., PMS database 114) included within/coupled to the practice management system (e.g., practice management system 102).

Databases (e.g., PMS database 114) are structured systems used to store, organize, and manage data efficiently, enabling easy retrieval, updating, and analysis. There are several types of databases, each designed for specific use cases. Relational databases, such as MySQL, PostgreSQL, Microsoft SQL Server, and Oracle Database, store data in structured tables with rows and columns, making them ideal for applications requiring high consistency and complex querying. NoSQL databases, like MongoDB, Cassandra, Redis, and Couchbase, are more flexible and scalable, making them suitable for handling unstructured or semi-structured data in real-time applications. Cloud databases, such as Amazon RDS, Google Cloud Firestore, and Azure SQL Database, offer scalability and managed services, allowing businesses to store and process data without on-premises infrastructure. For analyzing relationships between data points, graph databases like Neo4j and Amazon Neptune excel, while time-series databases such as InfluxDB and TimescaleDB are optimized for handling time-stamped data, often used in IoT and monitoring applications. Object-oriented databases, including db4o and ObjectDB, are designed to store objects in programming environments, and hierarchical databases, like IBM IMS, use a tree-like structure for parent-child relationships. Simpler storage needs can be addressed by flat file databases, such as CSV files, which store data in plain text or spreadsheet formats. Each type of database serves specific requirements based on the nature of the data, scalability demands, and application functionality, ensuring efficient data management across various domains.

Obtaining contract information (e.g., contract information 112) from a practice management system (PMS) in an orthodontic practice may involve accessing detailed data about the financial and service agreements between the practice and its patients. This information typically includes patient details, treatment plans, financial terms, insurance information, payment schedules, and any outstanding balances. Orthodontic contracts often outline long-term treatment plans with recurring monthly payments, making accurate management of these agreements essential. By retrieving this information, the practice can effectively track payments, manage accounts receivable, and ensure proper revenue recognition in compliance with accounting standards, such as GAAP. It also allows for better communication with patients about their financial responsibilities, treatment milestones, or any necessary adjustments. Additionally, accessing contract information (e.g., contract information 112) may help coordinate insurance claims and verify patient coverage, simplifying the billing process. This integration between financial and clinical aspects ensures a seamless workflow, as contract details tie directly into scheduling, treatment progress, and patient management. For orthodontic practices, where treatments often span months or years, efficiently managing contract information (e.g., contract information 112) may be critical for financial stability, patient satisfaction, and adherence to professional standards.

When obtaining 208 on the intermediate platform (e.g., intermediate platform 100) contract information (e.g., contract information 112) from the practice management system (e.g., practice management system 102), data transfer process 10 may periodically obtain 210 on the intermediate platform (e.g., intermediate platform 100) contract information (e.g., contract information 112) from the practice management system (e.g., practice management system 102).

To obtain 210 contract information (e.g., contract information 112) from a practice management system (PMS) on a periodic basis means regularly retrieving and reviewing this data at scheduled intervals, such as daily, weekly, or monthly, to ensure that the contract information (e.g., contract information 112) remain accurate and up to date. This process may involve consistently monitoring patient contracts, payment statuses, and treatment progress to identify any discrepancies, outstanding balances, or updates needed to the agreements. Performing this task periodically helps the practice maintain financial health by ensuring timely recognition of revenue and accurate tracking of accounts receivable.

In addition, a periodic review of contract information (e.g., contract information 112) supports compliance with accounting and regulatory standards, such as GAAP, by ensuring that revenue and expenses are accurately matched to the services provided during a specific period. This is particularly important for orthodontic practices, where treatments and payments often extend over months or years. By conducting these reviews systematically, the practice can make informed decisions, optimize cash flow, and maintain a clear and organized financial overview.

Once obtained 208, data transfer process 10 may process 212 the contract information (e.g., contract information 112) to define a contract type, an initial contract payment and a plurality of anticipated contract payments. For example, assume that for an orthodontic patient John Smith (e.g., patient 116), this patient is receiving traditional braces for a total cost of $6,000, wherein the initial contract payment is $2,400 and the remaining $3,600 is payable over a 24 month period via 24 monthly payments of $150 each.

To process contract information (e.g., contract information 112) to define a contract type, an initial contract payment, and a plurality of anticipated contract payments means organizing and analyzing the details of a patient's financial agreement to accurately categorize the contract and structure its payments. This process involves identifying the contract type, such as whether the agreement is for full payment upfront, partial payments with installments, or insurance-based coverage. The initial contract payment refers to the upfront payment made by the patient at the start of the treatment, which often covers initial services like consultations or diagnostic imaging. The plurality of anticipated contract payments refers to the scheduled future payments that the patient is expected to make over the course of the treatment. These payments are typically spread out over several months or years, depending on the duration of the treatment. By processing this information, the practice can ensure accurate revenue recognition, track payments effectively, and maintain clear financial records. This structured approach also helps the practice comply with accounting standards, such as GAAP, and provides patients with transparency regarding their financial obligations. In orthodontic practices, where treatments are long-term and payment plans are often complex, processing contract information (e.g., contract information 112) in this way ensures that financial agreements are properly managed throughout the treatment period.

Accordingly and continuing with the above-stated example, data transfer process 10 may process 212 the contract information (e.g., contract information 112) and define the following for orthodontic patient John Smith (e.g., patient 116):
contract type of “traditional braces”
initial contract payment of “$2,400”
plurality of anticipated contract payments of “24 monthly payments of $150 each”.

Once processed 212, data transfer process 10 may waterfall 214 the initial contract payment (i.e., $2,400) and the plurality of anticipated contract payments (i.e., 24 monthly payments of $150 each) in accordance with GAAP to generate one or more GAAP-adhering journal entries (e.g., GAAP-adhering journal entries 118) concerning the plurality of anticipated contract payments (i.e., 24 monthly payments of $150 each).

Waterfalling the initial contract payment (i.e., $2,400) and the plurality of anticipated contract payments (i.e., 24 monthly payments of $150 each) in accordance with GAAP means recognizing revenue over time in a structured manner, based on the actual delivery of services, rather than when payments are received. This approach adheres to the revenue recognition principle under GAAP, which mandates that revenue should only be recognized when it is earned—i.e., when the service or product is actually delivered to the customer-rather than when payment is made. In the context of an orthodontic practice, where treatments typically span months or even years, payments are often made by patients on a recurring basis. Instead of recognizing the entire payment upfront, the practice must allocate and recognize revenue in proportion to the services provided at any given point in time.

The process of waterfalling involves categorizing the contract into smaller segments that align with the progress of the treatment or service delivery. For example, if a patient enters into a treatment contract for orthodontic services, such as braces, the orthodontist will define an expected total payment schedule, with a portion of the total fee being paid upfront and the rest spread out across several months. The initial payment may cover initial services such as consultations and fitting of the braces, but this does not immediately translate into recognized revenue. Instead, the revenue is deferred until the actual orthodontic services are delivered over the course of the treatment plan.

To implement waterfalling in accordance with GAAP, the practice would first record the initial payment as deferred revenue, which is a liability on the balance sheet, because the revenue has not yet been earned. For example, the orthodontist may receive an initial payment of $2,400, but only a small portion of the treatment services may have been provided at the time of payment. The journal entry would reflect an increase in cash and a corresponding liability for deferred revenue. As the treatment progresses, revenue is gradually recognized based on the service delivered. For instance, if the orthodontist provides an adjustment to the braces or completes a treatment phase, a portion of the deferred revenue is recognized as earned revenue. The journal entry for this might involve debiting the deferred revenue (reducing the liability) and crediting the revenue account to reflect the earned portion.

This process continues throughout the duration of the treatment, with each subsequent payment received from the patient being similarly allocated to recognized revenue in alignment with the delivery of orthodontic services. If the patient makes a $150 monthly payment, a portion of that payment will reduce the deferred revenue balance, and an appropriate amount will be recognized as revenue, reflecting the progress of treatment provided during that period. This ensures that the financial statements accurately reflect the income earned as the services are rendered, rather than reflecting all the income upfront when the payments are made, regardless of when the services are completed.

By waterfalling anticipated contract payments in this way, the orthodontic practice ensures that its revenue recognition aligns with the actual service delivery, maintaining consistency with GAAP standards. This method helps the practice accurately match revenue with the expenses incurred for providing the treatment, leading to a more accurate representation of its financial position and performance. Additionally, it provides transparency for both the practice and the patients, as it clarifies how payments are applied over time and when revenue is officially earned. Waterfalling also helps with compliance during audits and tax filings, as it ensures the revenue is recognized in the appropriate periods, thus mitigating the risk of recognizing too much or too little revenue at any given time.

Data transfer process 10 may provide 216 the one or more GAAP-adhering journal entries (e.g., GAAP-adhering journal entries 118) to the accounting system (e.g., accounting system 106).

A GAAP-adhering journal entry (e.g., GAAP-adhering journal entries 118) is a financial record entered into an accounting system (e.g., accounting system 106) that complies with the principles and standards set by Generally Accepted Accounting Principles (GAAP). In an orthodontics practice, these journal entries ensure that financial transactions are accurately recorded, reported, and recognized in alignment with GAAP rules, promoting consistency, transparency, and accountability in the practice's financial management. This is particularly important in the orthodontic field, where payments and revenue recognition are often complex due to the long-term nature of treatment contracts and the use of installment payment plans.

For example, if a patient enters a contract for orthodontic treatment with an upfront payment and subsequent monthly payments, a GAAP-adhering journal entry would properly account for these transactions. The upfront payment would typically be recorded as deferred revenue (a liability), reflecting that the service has not yet been fully provided.

As treatment progresses and services are delivered, portions of this deferred revenue are recognized as earned revenue in the accounting program through journal entries. Similarly, monthly payments received would also be allocated between deferred and earned revenue based on the progress of the treatment plan.

Such journal entries (e.g., GAAP-adhering journal entries 118) also incorporate key GAAP principles, such as the matching principle, which ensures that revenue is recognized in the same period as the expenses incurred to deliver the related service. By adhering to GAAP standards, the accounting system (e.g., accounting system 106) can provide accurate financial reports that reflect the true financial position and performance of the orthodontics practice, ensure compliance during audits, and enhance decision-making for the business.

Data transfer process 10 may maintain 218 a rules database (e.g., rules database 120) that defines a plurality of contract types (e.g., contract types 122) and one or more GAAP-adhering waterfalling rules (e.g., waterfalling rules 124) concerning the plurality of contract types (e.g., contract types 122).

As discussed above, databases (e.g., rules database 120) are structured systems used to store, organize, and manage data efficiently, enabling easy retrieval, updating, and analysis. There are several types of databases, each designed for specific use cases. Relational databases, such as MySQL, PostgreSQL, Microsoft SQL Server, and Oracle Database, store data in structured tables with rows and columns, making them ideal for applications requiring high consistency and complex querying. NoSQL databases, like MongoDB, Cassandra, Redis, and Couchbase, are more flexible and scalable, making them suitable for handling unstructured or semi-structured data in real-time applications. Cloud databases, such as Amazon RDS, Google Cloud Firestore, and Azure SQL Database, offer scalability and managed services, allowing businesses to store and process data without on-premises infrastructure. For analyzing relationships between data points, graph databases like Neo4j and Amazon Neptune excel, while time-series databases such as

InfluxDB and TimescaleDB are optimized for handling time-stamped data, often used in IoT and monitoring applications. Object-oriented databases, including db40 and ObjectDB, are designed to store objects in programming environments, and hierarchical databases, like IBM IMS, use a tree-like structure for parent-child relationships. Simpler storage needs can be addressed by flat file databases, such as CSV files, which store data in plain text or spreadsheet formats. Each type of database serves specific requirements based on the nature of the data, scalability demands, and application functionality, ensuring efficient data management across various domains.

Data transfer process 10 may access 220 the rules database (e.g., rules database 120) to associate the contract type defined within the contract information (e.g., contract information 112) with one of the plurality of contract types (e.g., contract types 122) defined within the rules database (e.g., rules database 120), thus defining a identified contract type (e.g., identified contract type 126).

Continuing with the above-stated example, the contract information (e.g., contract information 112) for orthodontic patient John Smith (e.g., patient 116) is as follows:
contract type of “traditional braces”
initial contract payment of “$2,400”
plurality of anticipated contract payments of “24 monthly payments of $150 each”.

Accordingly, the contract type defined within the contract information (e.g., contract information 112) for orthodontic patient John Smith (e.g., patient 116) is “traditional braces”. Therefore and when associating the contract type defined within the contract information (e.g., contract information 112) with one of the plurality of contract types (e.g., contract types 122) defined within the rules database (e.g., rules database 120), the identified contract type (e.g., identified contract type 126) selected from the plurality of contract types (e.g., contract types 122) defined within the rules database (e.g., rules database 120) would be “traditional braces”.

Data transfer process 10 may obtain 222 one or more GAAP-adhering waterfalling rules concerning the identified contract type (e.g., identified contract type 126), thus defining one or more identified waterfalling rules (e.g., identified waterfalling rules 128). Accordingly and continuing with the above-stated example, these identified waterfalling rules (e.g., identified waterfalling rules 128) may be GAAP-adhering waterfalling rules that apply to the contract type “traditional braces”.

Data transfer process 10 may apply 224 the one or more identified waterfalling rules (e.g., identified waterfalling rules 128) concerning the identified contract type (e.g., identified contract type 126) to the initial contract payment (i.e., $2,400) and the plurality of anticipated contract payments (i.e., 24 monthly payments of $150 each) to generate the one or more GAAP-adhering journal entries (e.g., GAAP-adhering journal entries 118). As discussed above, data transfer process 10 may provide 216 the one or more GAAP-adhering journal entries (e.g., GAAP-adhering journal entries 118) to the accounting system (e.g., accounting system 106).

Data transfer process 10 may process 226 the contract information (e.g., contract information 112) obtained from the practice management system (e.g., practice management system 102) to remove Personally Identifiable Information (e.g., PII 130) from the contract information (e.g., contract information 112).

Personally Identifiable Information (PII) refers to any information that can be used to identify an individual, either directly or indirectly. In the context of an orthodontics practice, PII includes sensitive data collected and managed as part of patient care, scheduling, billing, and communication. Examples of PII in this setting include patient names, dates of birth, Social Security numbers, addresses, phone numbers, and email addresses. It also encompasses financial details such as credit card numbers and bank account information, as well as health-related data like medical histories, treatment plans, and imaging records, which are further classified as Protected Health Information (PHI) under regulations such as the Health Insurance Portability and Accountability Act (HIPAA) in the United States. Additionally, insurance details, including policy numbers and provider information, fall under PII.

Proper management of PII is crucial for maintaining patient privacy, complying with legal requirements, and safeguarding against unauthorized access or data breaches. Orthodontic practices often use secure practice management systems to store and handle this data, implementing safeguards like encryption, access controls, and regular audits to ensure compliance and security. By handling PII responsibly, an orthodontics practice can protect patient trust, avoid legal penalties, and uphold its reputation for providing high-quality, secure care.

Data transfer process 10 may effectuate 228 a reconciliation procedure to ensure that changes made within the practice management system (e.g., practice management system 102) are replicated onto the accounting system (e.g., accounting system 106).

Effectuating a reconciliation procedure to ensure that changes made within the practice management system (e.g., practice management system 102) are replicated onto the accounting system (e.g., accounting system 106) means establishing a process to ensure that both systems consistently reflect accurate financial information. This involves regularly comparing and verifying the data between the PMS, where patient treatments, payments, and adjustments are tracked, and the accounting system, where financial transactions are recorded. The process typically begins with extracting data from both systems and verifying that each transaction in the PMS has a corresponding entry in the accounting system. Any discrepancies found are then addressed, either through adjustments to the data in the PMS or the accounting system. This ensures the two systems align and maintain accurate financial records. Regular reconciliation—whether daily, weekly, or monthly—prevents errors such as missed payments or inaccurate revenue reporting. By ensuring both systems are in sync, the procedure guarantees compliance with accounting standards and provides a clear audit trail for internal reporting, tax filings, and audits. In some cases, automation can streamline this process, allowing for automatic syncing of data between the systems, reducing the potential for human error and improving efficiency. Overall, effectuating a reconciliation procedure is essential for maintaining financial accuracy and transparency in the practice's financial management.

Accordingly, if the contract information (e.g., contract information 112) for orthodontic patient John Smith (e.g., patient 116) is changed within the practice management system (e.g., practice management system 102), data transfer process 10 will ensure that such changes are replicated onto the accounting system (e.g., accounting system 106) via the above-described replication process.

Further, data transfer process 10 may effectuate 230 a reconciliation procedure to ensure that changes made within the accounting system (e.g., accounting system 106) are replicated onto the practice management system (e.g., practice management system 102).

Effectuating a reconciliation procedure to ensure that changes made within the accounting system (e.g., accounting system 106) are replicated onto the practice management system (e.g., practice management system 102) means implementing a process to ensure consistency and accuracy in financial data across both systems, with updates made in the accounting system being properly reflected in the PMS. The accounting system typically handles financial transactions, such as revenue recognition, payment processing, and invoicing, while the PMS tracks patient information, treatment plans, appointments, and billing details. If adjustments, payments, or financial updates are made in the accounting system, they need to be mirrored in the PMS to ensure both systems reflect the same financial data. This reconciliation procedure ensures that patient payments, balances, and treatment charges recorded in the accounting system are accurately replicated in the PMS, which is crucial for maintaining accurate patient records and ensuring smooth financial and operational workflows.

The process usually involves extracting and comparing data from both systems to verify that updates in the accounting system, such as payments, adjustments, or invoicing changes, are accurately reflected in the PMS. If discrepancies are found, corrections are made to align the data between the systems. This might involve updating payment records, adjusting patient balances, or ensuring that invoices and charges match across both platforms. Regular reconciliation-whether performed daily, weekly, or monthly-ensures that both systems are synchronized and provides an accurate and complete view of patient financials, helping to prevent errors like missed payments or unrecorded adjustments. Additionally, this reconciliation supports compliance with accounting standards, ensures accurate financial reporting, and improves decision-making for the practice. Automating the process, where possible, can further streamline this procedure, reducing manual errors and improving efficiency.

Accordingly, if one of the journal entries (e.g., GAAP-adhering journal entries 118) for orthodontic patient John Smith (e.g., patient 116) is changed within the accounting system (e.g., accounting system 106), data transfer process 10 will ensure that such changes are replicated onto the practice management system (e.g., practice management system 102) via the above-described replication process.

### AR Platform

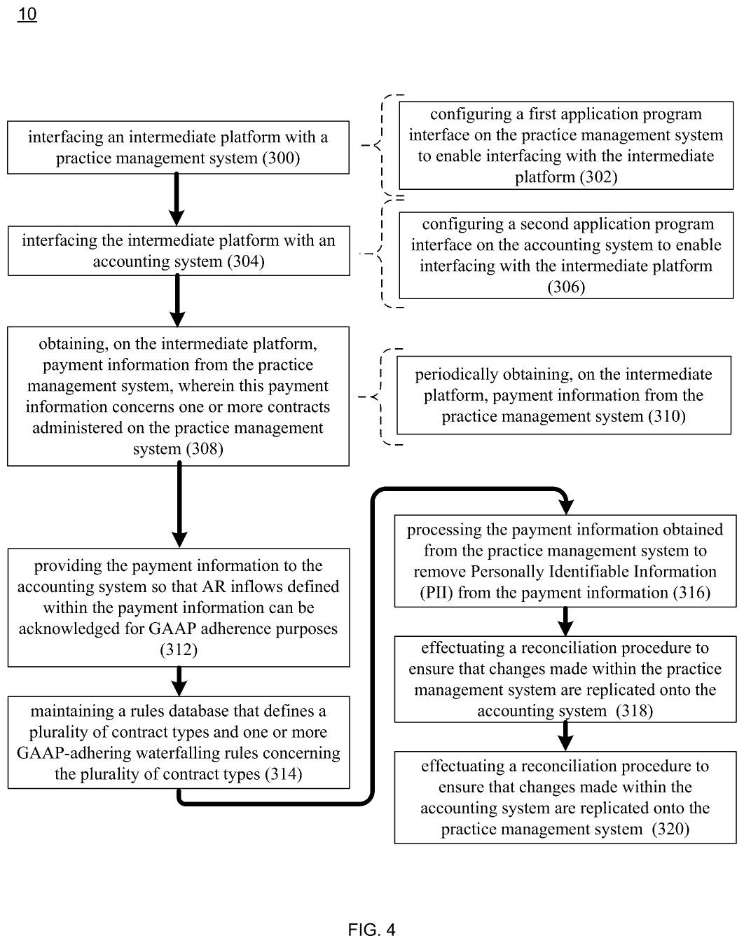

Referring also to FIG. 4 and as discussed above, data transfer process 10 may interface 300 an intermediate platform (e.g., intermediate platform 100) with a practice management system (e.g., practice management system 102). As also discussed above, a practice management system (e.g., practice management system 102) is a software platform designed to help healthcare practices, including medical practices, efficiently manage their administrative, financial, and operational tasks. It serves as a centralized tool for handling various aspects of practice management, such as patient scheduling, appointment reminders, billing, insurance claims processing, and record-keeping. By streamlining these processes, a PMS enables practices to enhance productivity, reduce administrative burden, and provide a seamless experience for both staff and patients.

As discussed above, an example of the practice management system (e.g., practice management system 102) may include but is not limited to an orthodontics practice management system (e.g., practice management system 102). As also discussed above, an orthodontics practice is a specialized healthcare facility focused on diagnosing, preventing, and treating dental and facial irregularities, primarily related to the alignment of teeth and jaws. Orthodontists, the dental specialists who operate these practices, provide treatments to correct issues such as misaligned teeth (malocclusion), overcrowding, gaps, overbites, underbites, and crossbites. The primary goal of an orthodontics practice is to improve the functionality, aesthetics, and overall health of a patient's smile.

As discussed above and when interfacing 300 an intermediate platform (e.g., intermediate platform 100) with a practice management system (e.g., practice management system 102), data transfer process 10 may configure 302 a first application program interface (e.g., first API 104) on the practice management system (e.g., practice management system 102) to enable interfacing 300 with the intermediate platform (e.g., intermediate platform 100). As also discussed above, an API (Application Programming Interface), in the context of a practice management system (PMS), is a set of tools and protocols that enable different software applications to communicate and interact with the PMS. APIs act as a bridge, allowing seamless data exchange and integration between the PMS and external systems, such as electronic health records (EHR), billing software, diagnostic tools, and patient portals.

As discussed above, data transfer process 10 may interface 304 the intermediate platform (e.g., intermediate platform 100) with an accounting system (e.g., accounting system 106). As also discussed above, an accounting system (e.g., accounting system 106) in the context of an orthodontic practice is specialized software designed to manage the financial and accounting functions of the practice efficiently. It provides tools to handle tasks such as bookkeeping, revenue tracking, expense management, billing, payroll, and financial reporting. By automating these processes, an accounting system (e.g., accounting system 106) helps orthodontic practices maintain accurate financial records, ensure compliance with regulations, and make informed decisions to improve financial health.

As discussed above and when interfacing 304 the intermediate platform (e.g., intermediate platform 100) with an accounting system (e.g., accounting system 106), data transfer process 10 may configure 306 a second application program interface (e.g., second API 110) on the accounting system (e.g., accounting system 106) to enable interfacing 304 with the intermediate platform (e.g., intermediate platform 100). As also discussed above, an API (Application Programming Interface), in the context of interfacing with an accounting system (e.g., accounting system 106), is a set of protocols and tools that enable different software applications to communicate with the accounting system. It acts as a bridge, allowing data to flow seamlessly between the accounting software and other systems, such as practice management systems, payment processing platforms, customer relationship management (CRM) tools, or payroll systems. This integration facilitates automated data sharing and enhances the efficiency and accuracy of financial processes.

Data transfer process 10 may obtain 308, on the intermediate platform (e.g., intermediate platform 100), payment information (e.g., payment information 132) from the practice management system (e.g., practice management system 102), wherein this payment information (e.g., payment information 132) concerns one or more contracts administered on the practice management system (e.g., practice management system 102). This payment information (e.g., payment information 132) may be extracted from the above-described contract information (e.g., contract information 112). This payment information (e.g., payment information 132) and/or contract information (e.g., contract information 112) may be obtained from a database (e.g., PMS database 114) included within/coupled to the practice management system (e.g., practice management system 102).

When obtaining 308, on the intermediate platform (e.g., intermediate platform 100), payment information (e.g., payment information 132) from the practice management system (e.g., practice management system 102), data transfer process 10 may periodically obtain 310, on the intermediate platform (e.g., intermediate platform 100), payment information (e.g., payment information 132) from the practice management system (e.g., practice management system 102). Obtaining 310 payment information (e.g., payment information 132) from a practice management system (PMS) on a periodic basis means regularly retrieving and reviewing this data at scheduled intervals, such as daily, weekly, or monthly, to ensure that the payment information (e.g., payment information 132) remains accurate and up to date. This process may involve consistently monitoring patient contracts, payment statuses, and treatment progress to identify any discrepancies, outstanding balances, or updates needed to the agreements. Performing this task periodically helps the practice maintain financial health by ensuring timely recognition of revenue and accurate tracking of accounts receivable.

Data transfer process 10 may provide 312 the payment information (e.g., payment information 132) to the accounting system (e.g., accounting system 106) so that AR inflows defined within the payment information (e.g., payment information 132) may be acknowledged for GAAP adherence purposes. For example, this payment information (e.g., payment information 132) provided to the accounting system (e.g., accounting system 106) may be utilizable to reconcile AR inflows defined within the payment information (e.g., payment information 132) with cash inflows defined within an external bank account (e.g., external bank account 134).

As discussed above, data transfer process 10 may maintain 314 a rules database (e.g., rules database 120) that defines a plurality of contract types (e.g., contract types 122) and one or more GAAP-adhering waterfalling rules (e.g., waterfalling rules 124) concerning the plurality of contract types (e.g., contract types 122). Accordingly, such a rules database (e.g., rules database 120) may enable data transfer process 10 to reconcile the AR inflows defined within the payment information (e.g., payment information 132) with cash inflows defined within the external bank account (e.g., external bank account 134) in accordance with GAAP policies and rules.

For example, assume that the above-described orthodontics practice received a $200 AR inflow on March 1st, a $350 AR inflow on March 2nd, and a $400 AR inflow and a $175 AR inflow on March 3rd. These discrete AR inflows may be included/defined within payment information 132 received from practice management system 102. Further, assume for this example that external bank account 134 shows a cash inflow of $200 on March 2nd, a cash inflow of $350 on March 3rd and cash inflows of $400 and $175 on March 4th. Accordingly, data transfer process 10 may reconcile these four AR inflows defined within practice management system 102 with these four cash inflows defined within the external bank account (e.g., external bank account 134).

Data transfer process 10 may process 316 the payment information (e.g., payment information 132) obtained from the practice management system (e.g., practice management system 102) to remove Personally Identifiable Information (e.g., PII 130) from the payment information (e.g., payment information 132). As discussed above, Personally Identifiable Information (PII) refers to any information that can be used to identify an individual, either directly or indirectly. In the context of an orthodontics practice, PII includes sensitive data collected and managed as part of patient care, scheduling, billing, and communication. Examples of PII in this setting include patient names, dates of birth, Social Security numbers, addresses, phone numbers, and email addresses.

As discussed above, data transfer process 10 may effectuate 318 a reconciliation procedure to ensure that changes made within the practice management system (e.g., practice management system 102) are replicated onto the accounting system (e.g., accounting system 106). As discussed above, effectuating a reconciliation procedure to ensure that changes made within the practice management system (e.g., practice management system 102) are replicated onto the accounting system (e.g., accounting system 106) means establishing a process to ensure that both systems consistently reflect accurate financial information. This involves regularly comparing and verifying the data between the PMS, where patient treatments, payments, and adjustments are tracked, and the accounting system, where financial transactions are recorded.

As discussed above, data transfer process 10 may effectuate 320 a reconciliation procedure to ensure that changes made within the accounting system (e.g., accounting system 106) are replicated onto the practice management system (e.g., practice management system 102). As discussed above, effectuating a reconciliation procedure to ensure that changes made within the accounting system (e.g., accounting system 106) are replicated onto the practice management system (e.g., practice management system 102) means implementing a process to ensure consistency and accuracy in financial data across both systems, with updates made in the accounting system being properly reflected in the PMS.

### General

As will be appreciated by one skilled in the art, the present disclosure may be embodied as a method, a system, or a computer program product. Accordingly, the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, the present disclosure may take the form of a computer program product on a computer-usable storage medium having computer-usable program code embodied in the medium.

Any suitable computer usable or computer readable medium may be utilized. The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, RF, etc.

Computer program code for carrying out operations of the present disclosure may be written in an object oriented programming language such as Java, Smalltalk, C++ or the like. However, the computer program code for carrying out operations of the present disclosure may also be written in conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network/a wide area network/the Internet (e.g., network 14).

The present disclosure is described with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, may be implemented by computer program instructions. These computer program instructions may be provided to a processor of a general purpose computer/special purpose computer/other programmable legal research processing apparatus, such that the instructions, which execute via the processor of the computer or other programmable legal research processing apparatus, create means for implementing the functions/acts specified in the flowchart and/or block diagram block or blocks.

These computer program instructions may also be stored in a computer-readable memory that may direct a computer or other programmable legal research processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable legal research processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

The flowcharts and block diagrams in the figures may illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustrations, and combinations of blocks in the block diagrams and/or flowchart illustrations, may be implemented by special purpose hardware-based systems that perform the specified functions or acts, or combinations of special purpose hardware and computer instructions.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

A number of implementations have been described. Having thus described the disclosure of the present application in detail and by reference to embodiments thereof, it will be apparent that modifications and variations are possible without departing from the scope of the disclosure defined in the appended claims.

What is claimed is:

1. A computer-implemented method, executed on a computing device, comprising:
interfacing an intermediate platform with a practice management system;
interfacing the intermediate platform with an accounting system;
obtaining, on the intermediate platform, payment information from the practice management system, wherein this payment information concerns one or more contracts administered on the practice management system; and
providing the payment information to the accounting system so that AR inflows defined within the payment information can be acknowledged for GAAP adherence purposes.

2. The computer-implemented method of claim 1 wherein interfacing an intermediate platform with a practice management system includes:
configuring a first application program interface on the practice management system to enable interfacing with the intermediate platform.

3. The computer-implemented method of claim 1 wherein interfacing the intermediate platform with an accounting system includes:
configuring a second application program interface on the accounting system to enable interfacing with the intermediate platform.

4. The computer-implemented method of claim 1 wherein the practice management system includes:
an orthodontics practice management system.

5. The computer-implemented method of claim 1 wherein the payment information provided to the accounting system is utilizable to reconcile AR inflows defined within the payment information with cash inflows defined within an external bank account.

6. The computer-implemented method of claim 1 wherein obtaining, on the intermediate platform, payment information from the practice management system includes:
periodically obtaining, on the intermediate platform, payment information from the practice management system.

7. The computer-implemented method of claim 1 further comprising:
maintaining a rules database that defines a plurality of contract types and one or more GAAP-adhering waterfalling rules concerning the plurality of contract types.

8. The computer-implemented method of claim 1 further comprising:
processing the payment information obtained from the practice management system to remove Personally Identifiable Information (PII) from the payment information.

9. The computer-implemented method of claim 1 further comprising:
effectuating a reconciliation procedure to ensure that changes made within the practice management system are replicated onto the accounting system.

10. The computer-implemented method of claim 1 further comprising:
effectuating a reconciliation procedure to ensure that changes made within the accounting system are replicated onto the practice management system.

11. A computer program product residing on a computer readable medium having a plurality of instructions stored thereon which, when executed by a processor, cause the processor to perform operations comprising:
interfacing an intermediate platform with a practice management system;
interfacing the intermediate platform with an accounting system;
obtaining, on the intermediate platform, payment information from the practice management system, wherein this payment information concerns one or more contracts administered on the practice management system; and
providing the payment information to the accounting system so that AR inflows defined within the payment information can be acknowledged for GAAP adherence purposes.

12. The computer program product of claim 11 wherein interfacing an intermediate platform with a practice management system includes:
configuring a first application program interface on the practice management system to enable interfacing with the intermediate platform.

13. The computer program product of claim 11 wherein interfacing the intermediate platform with an accounting system includes:
configuring a second application program interface on the accounting system to enable interfacing with the intermediate platform.

14. The computer program product of claim 11 wherein the practice management system includes:
an orthodontics practice management system.

15. The computer program product of claim 11 wherein the payment information provided to the accounting system is utilizable to reconcile AR inflows defined within the payment information with cash inflows defined within an external bank account.

16. The computer program product of claim 11 wherein obtaining, on the intermediate platform, payment information from the practice management system includes:
periodically obtaining, on the intermediate platform, payment information from the practice management system.

17. The computer program product of claim 11 further comprising:
maintaining a rules database that defines a plurality of contract types and one or more GAAP-adhering waterfalling rules concerning the plurality of contract types.

18. The computer program product of claim 11 further comprising:
processing the payment information obtained from the practice management system to remove Personally Identifiable Information (PII) from the payment information.

19. The computer program product of claim 11 further comprising:
effectuating a reconciliation procedure to ensure that changes made within the practice management system are replicated onto the accounting system.

20. The computer program product of claim 11 further comprising:
effectuating a reconciliation procedure to ensure that changes made within the accounting system are replicated onto the practice management system.

21. A computing system including a processor and memory configured to perform operations comprising:
interfacing an intermediate platform with a practice management system;
interfacing the intermediate platform with an accounting system;
obtaining, on the intermediate platform, payment information from the practice management system, wherein this payment information concerns one or more contracts administered on the practice management system; and
providing the payment information to the accounting system so that AR inflows defined within the payment information can be acknowledged for GAAP adherence purposes.

22. The computing system of claim 21 wherein interfacing an intermediate platform with a practice management system includes:
configuring a first application program interface on the practice management system to enable interfacing with the intermediate platform.

23. The computing system of claim 21 wherein interfacing the intermediate platform with an accounting system includes:
configuring a second application program interface on the accounting system to enable interfacing with the intermediate platform.

24. The computing system of claim 21 wherein the practice management system includes:
an orthodontics practice management system.

25. The computing system of claim 21 wherein the payment information provided to the accounting system is utilizable to reconcile AR inflows defined within the payment information with cash inflows defined within an external bank account.

26. The computing system of claim 21 wherein obtaining, on the intermediate platform, payment information from the practice management system includes:
periodically obtaining, on the intermediate platform, payment information from the practice management system.

27. The computing system of claim 21 further comprising:
maintaining a rules database that defines a plurality of contract types and one or more GAAP-adhering waterfalling rules concerning the plurality of contract types.

28. The computing system of claim 21 further comprising:
processing the payment information obtained from the practice management system to remove Personally Identifiable Information (PII) from the payment information.

29. The computing system of claim 21 further comprising:
effectuating a reconciliation procedure to ensure that changes made within the practice management system are replicated onto the accounting system.

30. The computing system of claim 21 further comprising:
effectuating a reconciliation procedure to ensure that changes made within the accounting system are replicated onto the practice management system.
